(19) United States
(12) Patent Application Publication
(10) Pub. No.: US 2022/0031996 A1
(43) Pub. Date: Feb. 3, 2022

(54) NOISE CANCELLATION FOR BALANCE PROSTHESIS

(71) Applicant: Cochlear Limited, Macquarie University, NSW (AU)

(72) Inventors: Koen Erik Van den Heuvel, Mechelen (BE); Joerg Pesch, Mechelen (BE)

(21) Appl. No.: 17/299,570

(22) PCT Filed: Apr. 27, 2020
(86) PCT No.: PCT/IB2020/053954

§ 371 Date: Jun. 3, 2021

Related U.S. Application Data
(60) Provisional application No. 62/844,295, filed on May 7, 2019.

(51) Int. Cl. A61M 21/00

(52) U.S. Cl. CPC A61M 21/00; A61M 2205/0244; A61M 2205/3327; A61M 2205/42; A61M 2021/0005; A61M 2205/04

(57) ABSTRACT

A system is provided which includes at least one first sensor subsystem configured to be worn on or implanted within a recipient's head and to generate first signals indicative of motion of the head and vibrational noise experienced by the recipient. The system further includes at least one second sensor subsystem spaced from the at least one first sensor subsystem. The second sensor subsystem is configured to generate second signals at least partially indicative of the vibrational noise experienced by the recipient. The system further includes signal processing circuitry configured to receive the first signals and the second signals, to filter the first signals in response at least in part to the second signals, and to generate third signals indicative of the motion of the head.

### BACKGROUND

### Field

The present application relates generally to balance prostheses configured to supplement or assist the vestibular system of a recipient, and more specifically to systems and methods for cancelling noise contributions from balance prostheses.

### Description of the Related Art

The vestibular system, located in the inner ear and including the three semicircular canals and the otolithic organs, provides a person with the sensation of angular and linear motion. Neural signals corresponding to this sensed motion are used by the brain to assist in a variety or processes including balance and determining orientation, and in related motor activities such as walking, standing, and visual orientation. Various abnormalities of the vestibular system are known (e.g., Meniere's disease), and in severe cases, can result in significant disability for those so afflicted.

Various forms of medical prostheses (e.g., balance prostheses) are configured to supplement or assist the vestibular system of a recipient. Examples of such balance prostheses have previously been disclosed (see, e.g., U.S. Pat. Nos. 9,089,692; 8,644,944; 6,546,291). Some balance prostheses can be wholly external to the recipient (e.g., worn by the recipient), while others can be partially implantable or wholly implantable (e.g., “mostly implantable,” “fully implantable,” or “totally implantable”) on or within the recipient. Such wholly implantable prostheses have the advantage of allowing the user to have a superior aesthetic result, as the recipient is visually indistinguishable in day-to-day activities from individuals that have not received such prostheses. Such wholly implantable prostheses also have a further advantage in generally being inherently waterproof, allowing the recipient to shower, swim, and so forth without needing to take any special measures. Furthermore, for recipients also suffering from some level of hearing loss, the balance prosthesis can be incorporated with an auditory prosthesis (e.g., cochlear implants) configured to facilitate hearing by the recipient.

### SUMMARY

In one aspect disclosed herein, a system is provided which comprises at least one first sensor subsystem configured to be worn on or implanted within a recipient's head and to generate first signals indicative of motion of the head and vibrational noise experienced by the recipient. The system further comprises at least one second sensor subsystem spaced from the at least one first sensor subsystem. The second sensor subsystem is configured to generate second signals at least partially indicative of the vibrational noise experienced by the recipient. The system further comprises signal processing circuitry configured to receive the first signals and the second signals, to filter the first signals in response at least in part to the second signals, and to generate third signals indicative of the motion of the head.

In another aspect disclosed herein, a method is provided which comprises receiving first signals having a first component indicative of first movements of a head of a recipient in a first frequency range and a second component indicative of second movements of the head in a second frequency range. The method further comprises receiving second signals at least partially indicative of the second movements. The method further comprises generating third signals by adaptively filtering the first signals in response, at least in part, to the second signals, the third signals indicative of the first signals with the second component suppressed.

In still another aspect disclosed herein, an apparatus is provided which comprises receiving at least one first orientation signal from a first sensor array comprising at least one first accelerometer. The at least one first orientation signal is indicative of a direction of gravity relative to a first coordinate system of the first sensor array. The method further comprises receiving at least one second orientation signal from a second sensor array comprising at least one second accelerometer spaced from the at least one first accelerometer. The at least one second orientation signal is indicative of the direction of gravity relative to a second coordinate system of the second sensor array. The method further comprises determining, in response to the at least one first orientation signal and the at least one second orientation signal, a relative orientation between the first coordinate system and the second coordinate system. The method further comprises using the relative orientation to transform first motion signals received from the first sensor array indicative of motion of the first sensor array relative to the first coordinate system and/or to transform second motion signals received from the second sensor array indicative of motion of the second sensor array relative to the second coordinate system such that the first motion signals and the second motion signals correspond to motions of the first sensor array and the second sensor array, respectively, relative to a common coordinate system.

### BRIEF DESCRIPTION OF THE DRAWINGS

Embodiments are described herein in conjunction with the accompanying drawings, in which:

FIG. 1 schematically illustrates an example system in accordance with certain embodiments described herein;

FIGS. 2A and 2B schematically illustrate an example first sensor subsystem and an example second sensor subsystem, respectively, in accordance with certain embodiments described herein;

FIGS. 3A and 3B schematically illustrate two example configurations of the first sensor subsystem and the second sensor subsystem in accordance with certain embodiments described herein;

FIGS. 4A and 4B schematically illustrate example signal processing circuitry in accordance with certain embodiments described herein;

FIG. 5 is a flow diagram of an example method in accordance with certain embodiments described herein;

FIG. 6 schematically illustrates an example set of first coordinate axes and an example set of second coordinate axes in accordance with certain embodiments described herein; and

FIG. 7 is a flow diagram of an example method in accordance with certain embodiments described herein.

### DETAILED DESCRIPTION

Certain embodiments described herein provide a system and method for improving the performance of a balance prosthesis. The balance prosthesis includes an input sensor subsystem configured to generate data signals indicative of motion of the head and environmental (e.g., vibrational) noise experienced by the recipient and a noise sensor subsystem configured to generate signals at least partially indicative of the environmental noise. The noise sensor subsystem can advantageously be spaced away from the input sensor subsystem (e.g., on a different portion of the recipient's body; spaced away from the recipient's body). The balance prosthesis can further include signal processing circuitry for filtering (e.g., adaptively filtering) the data signals received by the balance prostheses to mitigate (e.g., avoid; reduce; suppress; inhibit; cancel; remove; filter out) the effects of the environmental noise and/or other unwanted contributions to the data signals (e.g., artifacts, noise, oscillations, and/or instabilities) that could otherwise degrade performance of the balance prosthesis. The signal processing circuitry is configured to estimate the unwanted contributions and to create a cancelation signal which is added to the data signals to mitigate (e.g., avoid; reduce; suppress; inhibit; cancel; remove; cancel out) these estimated contributions so they do not unduly affect the performance of the balance prosthesis.

The teachings detailed herein are applicable, in at least some embodiments, to any type of balance prostheses (e.g., wholly external, partially implantable, or wholly implantable) and can include “stand-alone” balance prostheses and/or balance prostheses integrated with one or more other devices or prostheses (e.g., auditory prostheses, example of which include but are not limited to: electro-acoustic electrical/acoustic systems, cochlear implant devices, implantable hearing aid devices, middle ear implant devices, bone conduction devices, active bone conduction devices, passive bone conduction devices, percutaneous bone conduction devices, transcutaneous bone conduction devices, Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components). Embodiments can include any type of balance prosthesis that can utilize the teachings detailed herein and/or variations thereof. In some embodiments, the teachings detailed herein and/or variations thereof can be utilized in other types of prostheses beyond balance prostheses.

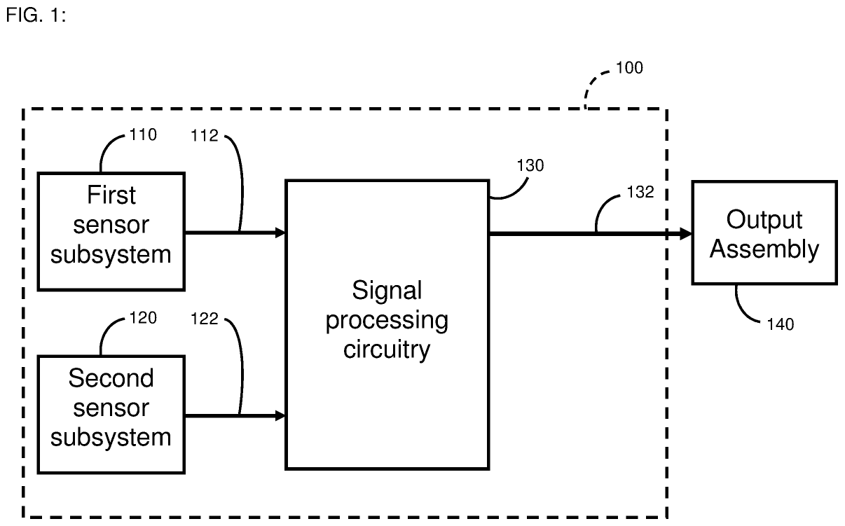

FIG. 1 schematically illustrates an example system 100 in accordance with certain embodiments described herein. The system 100 comprises at least one first sensor subsystem 110 configured to be worn on or implanted within a recipient's head and to generate first signals 112 indicative of motion of the head and vibrational noise experienced by the recipient. The system 100 further comprises at least one second sensor subsystem 120 spaced from the at least one first sensor subsystem 110. The at least one second sensor subsystem 120 is configured to generate second signals 122 that are at least partially indicative of the vibrational noise experienced by the recipient. The system 100 further comprises signal processing circuitry 130 configured to receive the first signals 112 and the second signals 122, to filter the first signals 112 in response at least in part to the second signals 122, and to generate third signals 132 indicative of the motion of the head.

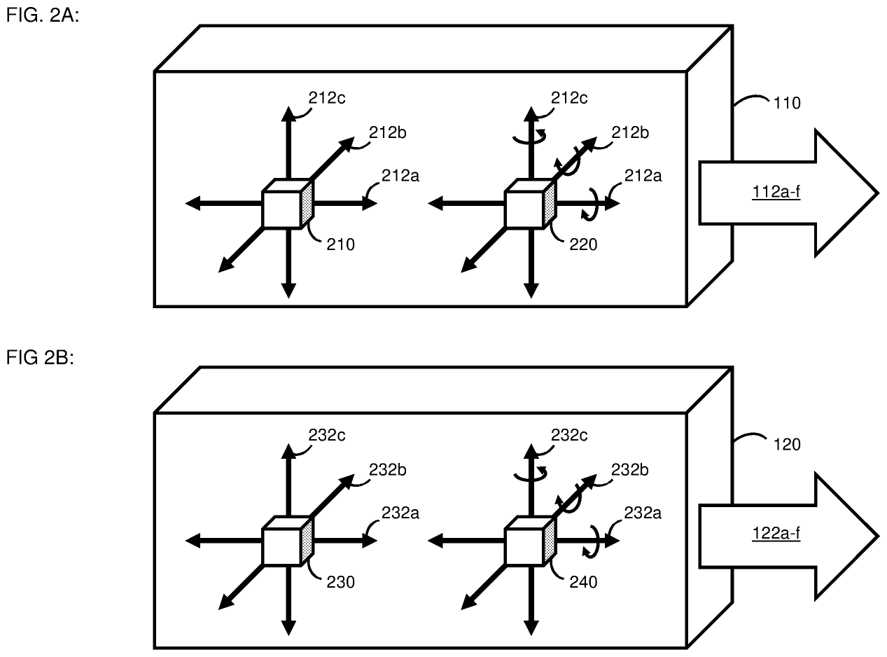

FIGS. 2A and 2B schematically illustrate an example first sensor subsystem 110 and an example second sensor subsystem 120, respectively, in accordance with certain embodiments described herein. The first sensor subsystem 110 (e.g., first sensor array) comprises at least one accelerometer 210 and/or at least one gyroscope 220, and the first signals 112 are at least partially indicative of accelerations (e.g., displacements) and/or rotations (e.g., angular velocities) of the first sensor subsystem 110 (e.g., of the head on which the first sensor subsystem 110 is worn or implanted). The second sensor subsystem 120 (e.g., second sensor array) comprises at least one accelerometer 230 and/or at least one gyroscope 240, and the second signals 122 are at least partially indicative of accelerations (e.g., translations; displacements) and/or rotations (e.g., angular velocities) of the second sensor subsystem 120. At least one of the at least one first sensor subsystem 110 and the at least one second sensor subsystem 120 comprises a plurality of microelectromechanical system (MEMS) sensors. Example accelerometers include, but are not limited to, MEMS quartz flexure suspension accelerometers available from a variety of vendors and example gyroscopes include, but are not limited to, MEMS oscillating mass gyroscopes available from a variety of vendors.

As shown in FIG. 2A, the example first sensor subsystem 110 comprises a three-axis accelerometer 210 sensitive to accelerations along three orthogonal sensing directions 212a-c (e.g., x-, y-, and z-coordinate axes of the first sensor subsystem 110) and a three-axis gyroscope 220 sensitive to rotations (e.g., roll, pitch, and yaw) about the three orthogonal sensing directions 212a-c. The three-axis accelerometer 210 is configured to generate first signals 110a-c indicative of the accelerations of the first sensor subsystem 110 (e.g., acceleration magnitudes and directions along each of the three coordinate axes) and the three-axis gyroscope 220 is configured to generate first signals 110d-f indicative of the rotations of the first sensor subsystem 110 (e.g., rotation magnitudes and directions about each of the three coordinate axes).

As shown in FIG. 2B, the example second sensor subsystem 120 comprises a three-axis accelerometer 230 sensitive to accelerations along three orthogonal sensing directions 232a-c (e.g., x-, y-, and z-coordinate axes of the second sensor subsystem 120) and a three-axis gyroscope 240 sensitive to rotations (e.g., roll, pitch, and yaw) about the three orthogonal sensing directions 232a-c. The three-axis accelerometer 230 is configured to generate second signals 120a-c indicative of the accelerations of the second sensor subsystem 120 (e.g., acceleration magnitudes and directions along each of the three coordinate axes) and the three-axis gyroscope 240 is configured to generate second signals 120d-f indicative of the rotations of the second sensor subsystem 120 (e.g., rotation magnitudes and directions about each of the three coordinate axes).

In certain embodiments, one or both of the at least one accelerometer 210 and the at least one accelerometer 230 comprises three single-axis accelerometers, each sensitive to accelerations along a respective single sensing direction (e.g., mounted within the respective sensor subsystem with the sensing directions orthogonal to one another), while in certain other embodiments, one or both of the at least one accelerometer 210 and the at least one accelerometer 230 comprises a single-axis accelerometer sensitive to accelerations along a single sensing direction and a two-axis accelerometer sensitive to accelerations along two sensing directions (e.g., mounted within the respective sensor subsystem with the sensing directions orthogonal to one another). In certain embodiments, one or both of the at least one gyroscope 220 and the at least one gyroscope 240 comprises three single-axis gyroscopes, each sensitive to rotations about a respective single sensing direction (e.g., mounted within the respective sensor subsystem with the sensing directions orthogonal to one another), while in certain other embodiments, one or both of the at least one gyroscope 220 and the at least one gyroscope 240 comprises a single-axis gyroscope sensitive to rotations about a single sensing direction and a two-axis gyroscope sensitive to rotations about two sensing directions (e.g., mounted within the respective sensor subsystem with the sensing directions orthogonal to one another). In certain embodiments, the at least one accelerometer 210 and the at least one gyroscope 220 of the first sensor subsystem 110 are identical to the at least one accelerometer 230 and the at least one gyroscope 240 of the second sensor subsystem 120, while in certain other embodiments, the at least one accelerometer 210 differs from the at least one accelerometer 230 and/or the at least one gyroscope 220 differs from the at least one gyroscope 240.

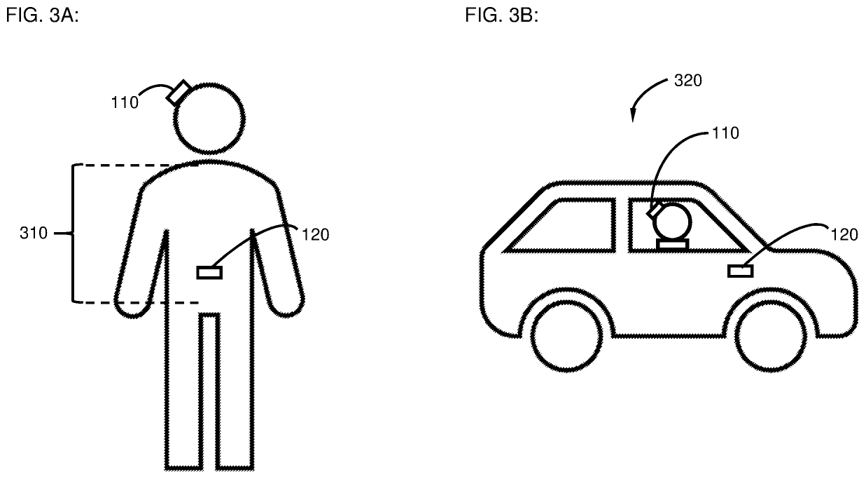

FIGS. 3A and 3B schematically illustrate two example configurations of the first sensor subsystem 110 and the second sensor subsystem 120 in accordance with certain embodiments described herein. In each of FIGS. 3A and 3B, the first sensor subsystem 110 is worn on or implanted within the recipient's head (e.g., such that a position and an orientation of the first sensor subsystem 110 relative to the recipient's head remains fixed during operation of the system 100). For example, the first sensor subsystem 110 can comprise a housing that contains the at least one accelerometer 210 and the at least one gyroscope 220, the housing configured to be worn on the recipient's head. For another example, the first sensor subsystem 100 can comprise an implantable housing (e.g., comprising titanium and/or another biocompatible material) that contains the at least one accelerometer 210 and the at least one gyroscope 220, the implantable housing configured to be implanted subcutaneously within the recipient's head. In certain embodiments, the first sensor subsystem 110 is integrated with at least a portion of a hearing prosthesis (e.g., a cochlear implant) or another type of medical prosthesis. For example, the first sensor subsystem 110 can be contained within an external sound processing unit of a cochlear implant system or contained within an implanted portion of a cochlear implant system.

In certain embodiments, the second sensor subsystem 120 is worn or carried by the recipient below the recipient's neck (e.g., on the recipient's torso 310) and is configured to monitor environmental noise (e.g., vibrational noise) experienced by the recipient. For example, as schematically illustrated by FIG. 3A, the second sensor subsystem 120 can be affixed to (e.g., clipped on; pinned on) the recipient's clothing, affixed to or on the recipient's belt, carried within a pocket of the recipient's clothing, etc. In certain such embodiments, the second sensor subsystem 120 is worn or carried by the recipient such that a position and an orientation of the second sensor subsystem 120 relative to the recipient's torso 310 remains fixed during operation of the system 100. In certain other such embodiments, the second sensor subsystem 120 is configured to monitor changes of the position and/or orientation of the second sensor subsystem 120, at least some of the second signals 122 are indicative of such changes, and the signal processing circuitry 130 is configured to ascertain the current position and/or orientation of the second sensor subsystem 120 using the second signals 122.

In certain embodiments, the second sensor subsystem 120 can be mounted within an electronic device (e.g., smartphone; tablet) carried by the recipient (e.g., within a pocket of the recipient's clothing; within a purse carried by the recipient) and/or kept by the recipient in proximity to the recipient (e.g., on a tabletop at which the recipient is sitting), and the second sensor subsystem 120 is configured to monitor environmental noise (e.g., vibrational noise) experienced by the recipient. In certain such embodiments, the electronic device comprises at least one accelerometer and/or at least one gyroscope which serve as the at least one accelerometer 230 and/or at least one gyroscope 240 of the second sensor subsystem 120. In certain such embodiments, the second sensor subsystem 120 is configured to monitor changes of the position and/or orientation of the second sensor subsystem 120, at least some of the second signals 122 are indicative of such changes, and the signal processing circuitry 130 is configured to ascertain the current position and/or orientation of the second sensor subsystem 120 using the second signals 122.

In certain embodiments, the second sensor subsystem 120 is mounted on or within a vehicle 320 carrying the recipient and is configured to monitor environmental noise (e.g., vibrational noise) experienced by the recipient. For example, as schematically illustrated by FIG. 3B, the second sensor subsystem 120 can be affixed to a portion of the vehicle 320 (e.g., automobile; motorcycle; bicycle; boat; airplane) such that a position and an orientation of the second sensor subsystem 120 relative to the vehicle 320 remains fixed during operation of the system 100 (e.g., affixed to a portion of the frame of the vehicle 320). In certain such embodiments, the vehicle 320 comprises at least one accelerometer and/or at least one gyroscope which serve as the at least one accelerometer 230 and/or at least one gyroscope 240 of the second sensor subsystem 120.

In certain embodiments, the signal processing circuitry 130 receives the first signals 112 from the first sensor subsystem 110 via one or more wires or cables, receives the second signals 122 from the second sensor subsystem 120 via one or more wires or cables, and/or transmits the third signals 132 to the output assembly 140 via one or more wires or cables. In certain other embodiments, the signal processing circuitry 130 is configured to be in wireless communication with (e.g., via an inductive or a radio frequency wireless link) one or more of the first sensor subsystem 110, the second sensor subsystem 120, and the output assembly 140. For example, the signal processing circuitry 130 can be configured to: wirelessly receive the first signals 112 from the first sensor subsystem 110 (e.g., the first sensor subsystem 110 comprises a wireless transmitter configured to send the first signals 112 to a wireless receiver of the signal processing circuitry 130); wirelessly receive the second signals 122 from the second sensor subsystem 120 (e.g., the second sensor subsystem 120 comprises a wireless transmitter configured to send the second signals 122 to a wireless receiver of the signal processing circuitry 130); wirelessly transmit the third signals 132 to the output assembly 140 (e.g., the signal processing circuitry 130 comprises a wireless transmitter configured to send the third signals 132 to a wireless receiver of the output assembly 140.

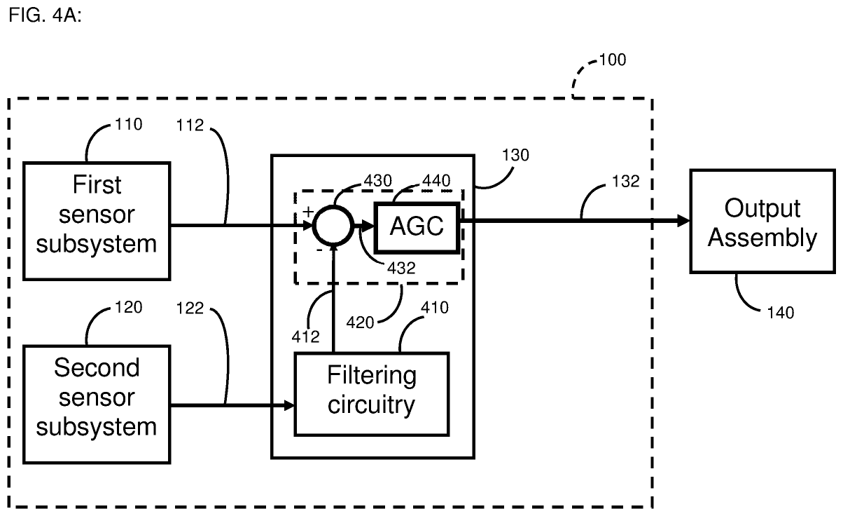

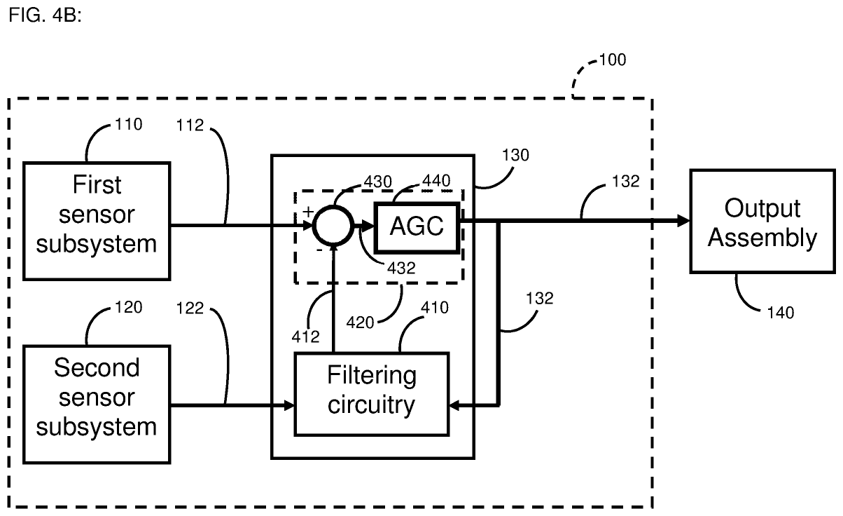

FIGS. 4A and 4B schematically illustrate example signal processing circuitry 130 in accordance with certain embodiments described herein. In certain embodiments, the signal processing circuitry 130 is integrated into the housing of the first sensor subsystem 110 or the housing of the second sensor subsystem 120, while in certain other embodiments, the signal processing circuitry 130 is in a housing separate from the housings of both the first sensor subsystem 110 and the second sensor subsystem 120. In certain embodiments, the signal processing circuitry 130 is a component of a signal processing unit (e.g., external or implantable) of a hearing prosthesis (e.g., a cochlear implant system). The signal processing circuitry 130 of certain embodiments comprises at least one processor (e.g., microelectronic circuitry; digital signal processor or DSP; application-specific integrated circuit or ASIC). In certain embodiments, the signal processing circuitry 130 further comprises at least one storage device (e.g., non-volatile memory; flash memory) operatively coupled to the at least one processor.

While FIGS. 1, 4A, and 4B schematically illustrates the signal processing circuitry 130 as comprising a single channel which receives the first signals 112 and the second signals 122, in certain embodiments, the signal processing circuitry 130 comprises a plurality of channels. For example, the first signals 112 can comprise three signals 112a-c corresponding to the displacements along each of the coordinate axes 212a-c detected by the at least one accelerometer 210 of the first sensor subsystem 110 and three signals 112d-f corresponding to the rotations about each of the coordinate axes 212a-c detected by the at least one gyroscope 220 of the first sensor subsystem 110. Further, the second signals 122 can comprise three signals 122a-c corresponding to the displacements along each of the coordinate axes 232a-c detected by the at least one accelerometer 230 of the second sensor subsystem 120 and three signals 122d-f corresponding to the rotations about each of the coordinate axes 232a-c detected by the at least one gyroscope 240 of the second sensor subsystem 120. The signal processing circuitry 130 can comprise six channels corresponding to six third signals 132a-f, each channel receiving one of the first signals 112a-f and the corresponding one of the second signals 122a-f and generating a corresponding one of the third signals 132a-f.

In certain embodiments, as schematically illustrated by FIGS. 4A and 4B, the signal processing circuitry 130 comprises filtering circuitry 410 configured to receive the second signals 122 and to generate filtering signals 412 in response at least in part to the second signals 122. The signal processing circuitry 130 of FIGS. 4A and 4B further comprises summation circuitry 420 configured to receive the first signals 112 and the filtering signals 412 and to generate the third signals 132 in response to the first signals 112 and the filtering signals 412. For example, the summation circuitry 420 can comprise an adder 430 and an automatic gain controller (“AGC”) 440. The adder 416 is configured to receive the first signals 112 from the first sensor subsystem 110 and the filtering signals 412 from the filtering circuitry 410 and to add the filtering signals 412 to the first signals 112 to generate resultant signals 432. The resultant signals 432 are received by the AGC 440 and represent net data signals (e.g., cleansed or clean signals) with a reduced noise component. The AGC 440 is configured to receive the resultant signals 432 and to further process the resultant signals 432 to generate the third signals 132 (e.g., processed data signals). The AGC 440 of certain embodiments comprises at least one processor (e.g., microelectronic circuitry) and at least one storage device (e.g., non-volatile memory; flash memory) operatively coupled to the at least one processor. The at least one processor comprises gain circuitry configured to adjust a gain applied to the resultant signals 432 and the at least one storage device comprises information (e.g., gain coefficient values) to be used by the at least one processor to adjust the applied gain.

As schematically illustrated by FIG. 4B, the filtering circuitry 410 of certain embodiments is further configured to provide adaptive filtering by receiving the third signals 132 and generating the filtering signals 412 in response to the second signals 122 and the third signals 132. For example, the filtering circuitry 410 can comprise one or more adjustable filters, such as, by way of example only and not by way of limitation, one or more adaptive filters (e.g., least-mean-square (LMS) adaptive filters; normalized least-mean-square (NLMS) adaptive filters; recursive least square (RLS) adaptive filters; filters utilizing an affine projection algorithm (APA); filters working in the time-domain; filters working in the frequency-domain). Other embodiments can be implemented using adjustable filters that are not adaptive filters. Any filtering configuration can enable the teachings detailed herein and/or variations thereof to be practiced can be utilized in at least some embodiments.

For example, the filtering circuitry 410 can utilize least-mean-square (LMS) adaptive filtering for adaptive noise cancelling, in which the motion of the head is represented as x₁(n), the vibrational noise is represented as x(n), the first signals 112 are represented as d(n), the filtering signals 412 are represented as y(n), and the third signals 132 are represented as e(n). Each iteration of the LMS filtering can include the following:

Filter output: y[n]=Σₙ₌₀^{N-1}x[n]w[n];

Estimation error: e[n]=d[n]=y[n];

Tap-weight adaptation: w[n+1]=w[n]+μx[n]e[n];

where a condition for stability can be 0<μ<2/(input signal power). Larger values for the step size can be used to increase the adaptation rate and/or to increase residual mean-squared error.

In certain embodiments, the signal processing circuitry 130 is configured to filter out noise contributions within a predetermined frequency range. For example, walking and running frequencies can be below 25 Hz, and the signal processing circuitry 130 can filter out all contributions to the first signals 112 that are above 25 Hz and can only filter out noise contributions indicated by the second signals 122 for frequencies less than 25 Hz. In certain embodiments, the signal processing circuitry 130 integrates the first signals 112 and/or integrates the second signals 122 over a predetermined integration time period, thereby effectively slowing the adaptation speed of the filtering circuitry 410 of the signal processing circuitry 130 and reducing the effects of artifacts of short time duration.

In certain embodiments, the output assembly 140 comprises an implanted stimulator or actuator, while in certain other embodiments, the output assembly 140 comprises an external stimulator or actuator. For example, U.S. Pat. No. 9,089,692 discloses an implantable vestibular stimulation device that is compatible with certain embodiments described herein, and U.S. Pat. No. 6,546,291 discloses an externally wearable stimulator device that is compatible with certain embodiments described herein. The output assembly 140 is configured to receive the third signals 132 and to communicate sensor signals to the recipient, the sensory signals generated in response to the third signals 132 and configured to be perceived by the recipient. For example, the sensory signals can directly stimulate a portion of the recipient's nervous system (e.g., vestibular system) which the recipient can perceive and respond to so as to maintain balance or can be optical, auditory, or tactile signals which the recipient can perceive and respond to so as to maintain balance.

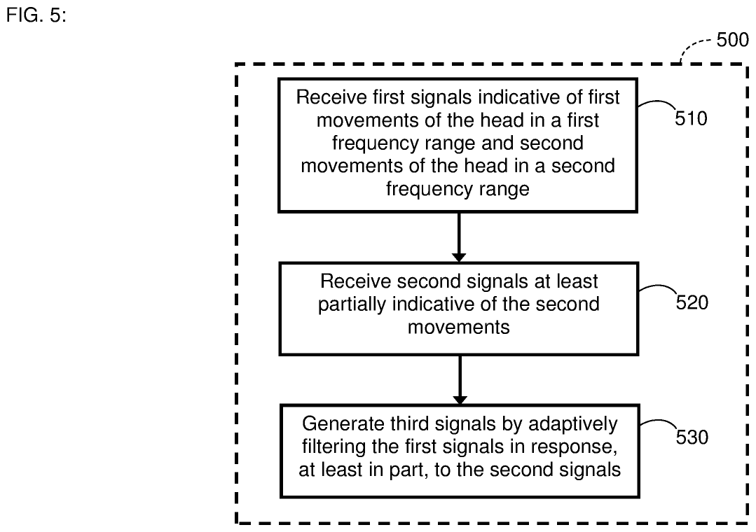

FIG. 5 is a flow diagram of an example method 500 in accordance with certain embodiments described herein. While the method 500 is described herein by referring to the example system 100 of FIGS. 1, 2A-2B, 3A-3B, and 4A-4B, other systems and components are also compatible with being used with the method 500 in accordance with certain embodiments described herein.

In an operational block 510, the method 500 comprises receiving first signals 112 (e.g., from a first sensor subsystem 110 worn on or implanted within a recipient's head) having a first component indicative of first movements of a head of a recipient in a first frequency range and a second component indicative of second movements of the head in a second frequency range. For example, the first frequency range can be in a range of less than or equal to 25 Hz and the second frequency range can be in a range of greater than 25 Hz. In certain embodiments, both the first movements and the second movements comprise translations and/or rotations of the head relative to three orthogonal axes (e.g., coordinate axes 212a-c).

In an operational block 520, the method 500 further comprises receiving second signals 122 (e.g., from a second sensor subsystem 120 spaced from the first sensor subsystem 110 and not worn on or implanted within the recipient's head) at least partially indicative of the second movements. In an operational block 530, the method 500 further comprises generating third signals 132 by adaptively filtering the first signals 112 in response, at least in part, to the second signals 122. The third signals 132 are indicative of the first signals 112 with the second component suppressed. In certain embodiments, the adaptive filtering is selected from the group consisting of: least-mean-square (LMS) filtering; normalized least-mean-square (NLMS) filtering; recursive least square (RLS) filtering; affine projection algorithm (APA) filtering. In certain embodiments, the filtering is performed in the time-domain, while in certain other embodiments, the filtering is performed in the frequency-domain.

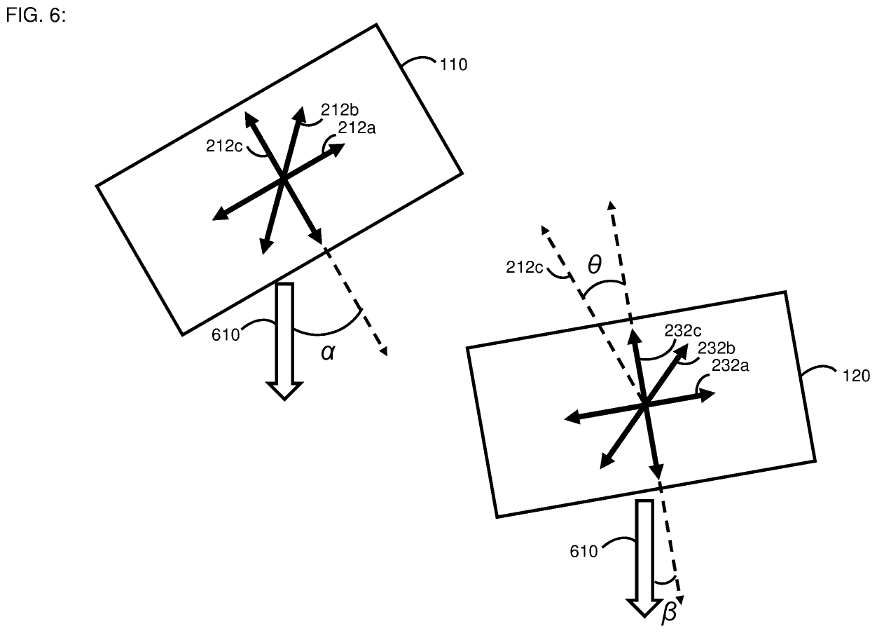

As schematically illustrated in FIGS. 2A-2B, in certain embodiments, the first signals 112 generated by the first sensor subsystem 110 are based on a first coordinate system having three orthogonal first coordinate axes 212a-c and the second signals 122 generated by the second sensor subsystem 120 are based on a second coordinate system having three orthogonal second coordinate axes 232a-c. By virtue of the spacing between the first sensor subsystem 110 and the second sensor subsystem 120, the second coordinate axes 232a-c have a different origin (e.g., position at which the three coordinate axes intersect one another) than do the first coordinate axes 212a-c. In certain embodiments, the second coordinate axes 232a-c also have a different orientation than do the first coordinate axes 212a-c (e.g., a non-zero orientation angle θ between the first coordinate axes 212a-c and the second coordinate axes 232a-c). FIG. 6 schematically illustrates an example set of first coordinate axes 212a-c and an example set of second coordinate axes 232a-c in accordance with certain embodiments described herein. In certain embodiments, the signal processing circuitry 130 is configured to apply a transformation to the first signals 112 and/or the second signals 122 such that the first signals 112 and the second signals 122 are indicative of motions of the first sensor subsystem 110 and the second sensor subsystem 120, respectively, relative to a common coordinate system (e.g., a common set of three orthogonal coordinate axes).

For example, the first sensor subsystem 110 and the second sensor subsystem 120 can be configured to be temporarily placed in a predetermined orientation relative to one another (e.g., the second coordinate axes 232a-c parallel to the first coordinate axes 212a-c). In certain such embodiments, the first sensor subsystem 110 and the second sensor subsystem 120 can be configured to mate with one another (e.g., using one or more corresponding protrusions, recesses, and/or edges of the first sensor subsystem 110 and the second sensor subsystem 120), such that the first sensor subsystem 110 and the second sensor subsystem 120 have the predetermined orientation relative to one another. Upon the first sensor subsystem 110 and the second sensor subsystem 120 being in the predetermined orientation with one another, the signal processing circuitry 130 can use the predetermined orientation to transform the first signals 112 and/or the second signals 122 to correspond to motions relative to a common coordinate system (e.g., a common set of three orthogonal coordinate axes). In addition, the signal processing circuitry 130 can use the first signals 112d-f from the at least one gyroscope 220 and/or the second signals 122d-f from the at least one gyroscope 240 to track rotations of the first sensor subsystem 110 and/or the second sensor subsystem 120 relative to one another and to update the relative orientation that is used to transform the first signals 112 and/or the second signals 122.

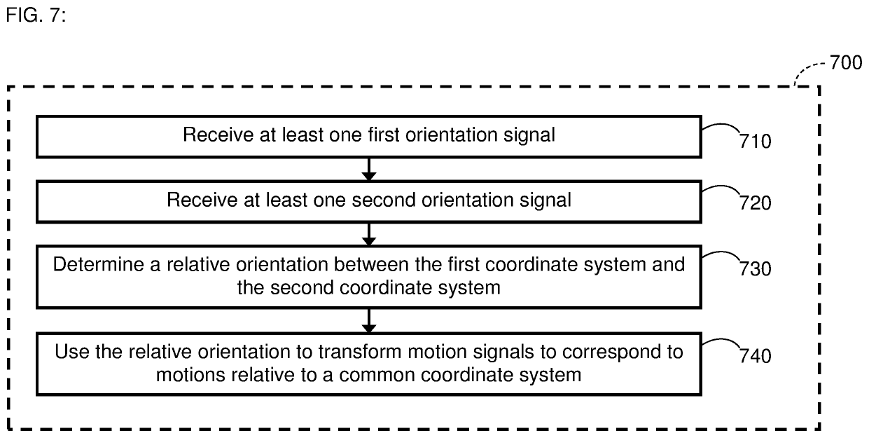

For another example, the signal processing circuitry 130 can utilize the direction of gravity 610 to determine the relative orientation between the first coordinate axes 212a-c and the second coordinate axes 232a-c. FIG. 7 is a flow diagram of an example method 700 in accordance with certain embodiments described herein. While the method 700 is described herein by referring to the example system 100 of FIGS. 1, 2A-2B, 3A-3B, 4A-4B, and 6, other systems and components are also compatible with being used with the method 700 in accordance with certain embodiments described herein. In certain embodiments, the method 700 can be combined with the example method 500 to correlate the first signals 112 with the second signals 122 such that a common set of coordinate axes are used for the first signals 112 and the second signals 122.

In an operational block 710, the method 700 comprises receiving (e.g., by the signal processing circuitry 130) at least one first orientation signal from the first sensor subsystem 110 (e.g., first sensor array) comprising at least one first accelerometer 210. The at least one first orientation signal is indicative of a direction of gravity 610 relative to a first coordinate system of the first sensor subsystem 110 (e.g., the direction of gravity 610 at an angle α relative to the coordinate axis 212c). In an operational block 720, the method 700 further comprises receiving (e.g., by the signal processing circuitry 130) at least one second orientation signal from the second sensor subsystem 120 (e.g., second sensor array) comprising at least one second accelerometer 230 spaced from the at least one first accelerometer 210. The at least one second orientation signal is indicative of the direction of gravity 610 relative to a second coordinate system of the second sensor substrate 120 (e.g., the direction of gravity 610 at an angle β relative to the coordinate axis 232c).

In an operational block 730, the method 700 further comprises determining (e.g., by the signal processing circuitry 130), in response to the at least one first orientation signal and the at least one second orientation signal, a relative orientation between the first coordinate system and the second coordinate system (e.g., the coordinate axis 212c at an angle θ=α−β relative to the coordinate axis 232c). In an operational block 740, the method 700 further comprises using (e.g., by the signal processing circuitry 130) the relative orientation to transform the first signals 112 received from the first sensor subsystem 110 indicative of motion of the first sensor subsystem 110 relative to the first coordinate system and/or to transform the second signals 122 received from the second sensor subsystem 120 indicative of motion of the second sensor subsystem 120 relative to the second coordinate system such that the first signals 112 and the second signals 122 correspond to motions of the first sensor subsystem 110 and the second sensor subsystem 120, respectively, relative to a common coordinate system.

In certain embodiments, the at least one first orientation signal is received while the first sensor subsystem 110 is stationary and the at least one second orientation signal is received while the second sensor subsystem 120 is stationary. For example, the at least one first orientation signal and the at least one second orientation signal can be received during an initial orientation procedure in which both the first sensor subsystem 110 and the second sensor subsystem 120 are held stationary. For another example, the at least one first orientation signal and the at least one second orientation signal can be received during subsequent orientation procedures in which both the first sensor subsystem 110 and the second sensor subsystem 120 are detected to be stationary. In certain other embodiments, the at least one first orientation signal and the at least one orientation signal are received while one or both of the first sensor subsystem 110 and the second sensor subsystem 120 are non-stationary (e.g., but while the relative orientation between the first sensor subsystem 110 and the second sensor subsystem 120 is unchanging). In certain embodiments, the at least one first orientation signal and the at least one second orientation signal are received concurrently with one another, while in certain other embodiments, the at least one first orientation signal and the at least one second orientation signal are received at different times from one another.

In certain embodiments, the method 700 further comprises monitoring motions of the first sensor subsystem 110 and the second sensor subsystem 120 to detect changes of the relative orientation, updating the relative orientation, and using the updated relative orientation to transform the first signals 112 and/or the second signals 122. For example, the first signals 112d-f from the at least one first gyroscope 220 are indicative of rotations of the first sensor subsystem 110 and the second signals 122d-f from the at least one second gyroscope 240 are indicative of rotations of the second sensor subsystem 120, and these first signals 112d-f and second signals 122d-f can be used to update the relative orientation of the first sensor subsystem 110 and the second sensor subsystem 120.

It is to be appreciated that the embodiments disclosed herein are not mutually exclusive and may be combined with one another in various arrangements.

The invention described and claimed herein is not to be limited in scope by the specific example embodiments herein disclosed, since these embodiments are intended as illustrations, and not limitations, of several aspects of the invention. Any equivalent embodiments are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example embodiments disclosed herein, but should be defined only in accordance with the claims and their equivalents.

1. A system comprising:
at least one first sensor subsystem configured to be worn on or implanted within a recipient's head and to generate first signals indicative of motion of the head and vibrational noise experienced by the recipient;
at least one second sensor subsystem spaced from the at least one first sensor subsystem, the second sensor subsystem configured to generate second signals at least partially indicative of the vibrational noise experienced by the recipient; and
signal processing circuitry configured to receive the first signals and the second signals, to filter the first signals in response at least in part to the second signals, and to generate third signals indicative of the motion of the head.

2. The system of claim 1, wherein at least one of the at least one first sensor subsystem and the at least one second sensor subsystem comprises a plurality of microelectromechanical system (MEMS) sensors.

3. The system of claim 1, wherein the at least one first sensor subsystem comprises at least one accelerometer and the first signals are at least partially indicative of accelerations of the head.

4. The system of claim 1, wherein the at least one first sensor subsystem comprises at least one gyroscope and the first signals are at least partially indicative of rotations of the head.

5. The system of claim 1, wherein the third signals are indicative of the motion of the head relative to three orthogonal axes.

6. The system of claim 1, wherein the at least one second sensor subsystem comprises at least one accelerometer and/or at least one gyroscope.

7. The system of claim 1, wherein the at least one second sensor subsystem is worn or carried by the recipient below the recipient's neck.

8. The system of claim 1, wherein the at least one second sensor subsystem is mounted within an electronic device carried by the recipient or within a vehicle carrying the recipient.

9. The system of claim 1, wherein the signal processing circuitry is configured to adaptively filter the first signals.

10. The system of claim 1, wherein the signal processing circuitry comprises:
filtering circuitry configured to receive the second signals and to generate filtering signals in response at least in part to the second signals; and
summation circuitry configured to receive the first signals and the filtering signals and to generate the third signals in response to the first signals and the filtering signals.

11. The system of claim 10, wherein the filtering circuitry is further configured to receive the third signals and to generate the filtering signals in response to the second signals and the third signals.

12. The system of claim 10, wherein the summation circuitry comprises:
an adder configured to add the filtering signals to the first signals to generate resultant signals; and
an automatic gain controller configured to apply a gain to the resultant signals to generate the third signals.

13. The system of claim 1, further comprising an output assembly configured to receive the third signals and to communicate sensory signals to the recipient, the sensory signals generated in response to the third signals and configured to be perceived by the recipient.

14. The system of claim 1, wherein the system comprises a balance prosthesis.

15. A method comprising:
receiving first signals having a first component indicative of first movements of a head of a recipient in a first frequency range and a second component indicative of second movements of the head in a second frequency range;
receiving second signals at least partially indicative of the second movements; and
generating third signals by adaptively filtering the first signals in response, at least in part, to the second signals, the third signals indicative of the first signals with the second component suppressed.

16. The method of claim 15, wherein both the first movements and second movements comprise translations and/or rotations of the head relative to three orthogonal axes.

17. The method of claim 15, wherein the filtering is selected from the group consisting of: least mean squares (LMS) filtering; normalized least mean squares (NLMS) filtering; recursive least squares (RLS) filtering; affine projection algorithm (APA) filtering.

18. The method of claim 15, wherein the first frequency range is less than or equal to 25 Hz and the second frequency range is greater than 25 Hz.

19. The method of claim 15, wherein the first signals are received from a first sensor subsystem worn on or implanted within a recipient's head, the second signals are received from second sensor subsystem spaced from the first sensor subsystem and not worn on or implanted within the recipient's head, the method further comprising determining a relative orientation of the first sensor subsystem to the second sensor subsystem.

20. A method comprising:
receiving at least one first orientation signal from a first sensor array comprising at least one first accelerometer, the at least one first orientation signal indicative of a direction of gravity relative to a first coordinate system of the first sensor array;
receiving at least one second orientation signal from a second sensor array comprising at least one second accelerometer spaced from the at least one first accelerometer, the at least one second orientation signal indicative of the direction of gravity relative to a second coordinate system of the second sensor array;
determining, in response to the at least one first orientation signal and the at least one second orientation signal, a relative orientation between the first coordinate system and the second coordinate system; and
using the relative orientation to transform first motion signals received from the first sensor array indicative of motion of the first sensor array relative to the first coordinate system and/or to transform second motion signals received from the second sensor array indicative of motion of the second sensor array relative to the second coordinate system such that the first motion signals and the second motion signals correspond to motions of the first sensor array and the second sensor array, respectively, relative to a common coordinate system.

21. The method of claim 20, wherein receiving the at least one first orientation signal is performed while the first sensor array is stationary and receiving the at least one second orientation signal is performed while the second sensor array is stationary.

22. The method of claim 20, wherein receiving the at least one first orientation signal and receiving the at least one second orientation signal are performed concurrently.

23. The method of claim 20, further comprising monitoring motions of the first sensor array and the second sensor array to detect changes of the relative orientation, updating the relative orientation, and using the updated relative orientation to transform the first motion signals and/or the second motion signals.

24. The method of claim 20, wherein the first sensor array further comprises at least one first gyroscope and the first motion signals received from the first sensor array are indicative of translations of the first sensor array along each of three orthogonal axes of the first coordinate system and of rotations of the first sensor array about each of the three orthogonal axes of the first coordinate system.

25. The method of claim 24, wherein the second sensor array further comprises at least one second gyroscope and the second motion signals received from the second sensor array are indicative of translations of the second sensor array along each of three orthogonal axes of the second coordinate system and of rotations of the second sensor array about each of the three orthogonal axes of the second coordinate system.

26. The method of claim 20, wherein the first sensor array is configured to be worn on or implanted within a recipient's head.

27. The method of claim 26, wherein the second sensor array is configured to be worn on or carried by a recipient spaced from the first sensor array or mounted on a vehicle carrying the recipient.
